(19) United States
(12) Patent Application Publication
(10) Pub. No.: US 2015/0355194 A1
(43) Pub. Date: Dec. 10, 2015

(54) MARKERS FOR LIPID METABOLISM

(71) Applicant: UNIVERSITY OF SOUTHERN CALIFORNIA, Los Angeles, CA (US)

(72) Inventor: Sean P. Curran, La Mirada, CA (US)

(21) Appl. No.: 14/622,543

(22) Filed: Feb. 13, 2015

Related U.S. Application Data
(60) Provisional application No. 61/940,167, filed on Feb. 14, 2014.

(51) Int. Cl. G01N 33/68; C12Q 1/68

(52) U.S. Cl. CPC G01N 33/6893; C12Q 1/6883; C12Q 2600/136; G01N 2800/044

(57) ABSTRACT

Disclosed herein is a method for treating obesity in a subject in need thereof. Also disclosed herein is a method for protecting a subject in need thereof from obesity. Further disclosed herein is a method for monitoring an efficacy of a treatment for obesity in a subject.

### CROSS-REFERENCE TO RELATED APPLICATIONS

This application claims priority to U.S. Provisional Patent Application No. 61/940,167, filed Feb. 14, 2014, the entire contents of which are hereby incorporated by reference.

### STATEMENT OF GOVERNMENT INTEREST

This invention was made with government support under contract number AG032308 awarded by the National Institutes of Health. The government has certain rights in the invention.

### TECHNICAL FIELD

The present invention relates to methods for treating and diagnosing obesity, heart disease, and other diseases associated with deregulated lipid homeostasis.

### BACKGROUND

The occurrence of obesity is increasing in the human population. For example, between 1980 and 2000, obesity rates have doubled in adults and since 1980, overweight rates have doubled and tripled among children and adolescents, respectively. Obesity leads to numerous health problems, including type 2 diabetes, heart disease and other metabolic syndromes associated with deregulated lipid homeostasis.

Animals, including humans, maintain metabolic homeostasis through the coordinated metabolism of available intracellular nutrients. When fasting or starving, animals switch their utilization of dietary carbohydrates to stored fats and proteins (i.e., amino acids) to satisfy energy requirements.

Accordingly, a need remains in the art for the identification of therapeutic targets that can be manipulated to promote the use of fats as an energy source and/or prevent the accumulation of stored fat, thereby treating obesity.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “control sample” or “control” as used herein means a sample or specimen taken from a subject, or an actual subject who does not have enhanced lipid metabolism.

“Nucleic acids” as used herein can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine, and isoguanine. Nucleic acids can be obtained by isolation or extraction methods, by chemical synthesis methods or by recombinant methods.

A “peptide,” “protein,” or “polypeptide” as used herein can mean a linked sequence of amino acids and can be natural, synthetic, or a modification or combination of natural and synthetic.

The term “sample,” “test sample,” “specimen,” “biological sample,” “sample from a subject,” or “subject sample” as used herein interchangeably, means a sample or isolate of blood, tissue, urine, serum, plasma, amniotic fluid, cerebrospinal fluid, placental cells or tissue, endothelial cells, leukocytes, or monocytes, can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

The term also means any biological material being tested for and/or suspected of containing an analyte of interest such as Nrf2/SKN-1 and MDT-15. The sample may be any tissue sample taken or derived from the subject. In some embodiments, the sample from the subject may comprise protein. In some embodiments, the sample from the subject may comprise nucleic acid. Any cell type, tissue, or bodily fluid may be utilized to obtain a sample. Such cell types, tissues, and fluid may include sections of tissues such as biopsy (such as muscle biopsy) and autopsy samples, frozen sections taken for histological purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include muscle tissue or fibres, lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

Methods well-known in the art for collecting, handling and processing muscle tissue or fibre, urine, blood, serum and plasma, and other body fluids, are used in the practice of the present disclosure. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary (e.g., most urine samples, a pre-processed archived sample, etc.), pretreatment of the sample is an option that can be performed for mere convenience (e.g., as part of a protocol on a commercial platform). The sample may be used directly as obtained from the subject or following pretreatment to modify a characteristic of the sample. Pretreatment may include extraction, concentration, inactivation of interfering components, and/or the addition of reagents.

The term “subject” or “patient” as used herein interchangeably, means any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc)) and a human. In some embodiments, the subject or patient may be a human or a non-human. The subject or patient may be undergoing other forms of treatment.

“Treat”, “treating” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an antibody or pharmaceutical composition of the present invention to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Variants can be a fragment thereof. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Method of Identifying a Factor for Enhancing Lipid Metabolism

Provided herein is a method of identifying factors and subfactors for enhancing lipid metabolism in a subject in need thereof. The method includes obtaining a sample from the subject and measuring or detecting a level of the factor in the sample either alone or in combination with one, two, three, or more factors. The method also includes measuring or detecting a level of a subfactor in the sample alone, in combination with the factor, in combination with one, two, three or more factors, in combination with one, two, three, or more subfactors, or any combination thereof.

A change in the level of the factor in the sample obtained from the subject relative to a control sample identifies the factor for enhancing lipid metabolism, thereby indicating that lipid metabolism is enhanced or increased in the subject. The change in the level of the factor may be an increase in or an up-regulation of the expression or activity of the factor in the sample obtained from the subject. Alternatively, the change in the level of the factor may be a decrease in the level of or an absence of the factor in the sample obtained from the subject. The change in the level of the factor may be a decrease or a down-regulation of the expression or activity of the factor in the sample obtained from the subject.

A change in the level of the subfactor in the sample obtained from the subject relative to the control sample identifies the subfactor for enhancing lipid metabolism, thereby indicating that lipid metabolism is enhanced or increased in the subject. The change in the level of the subfactor may be an increase in or an up-regulation of the expression or activity of the subfactor in the sample obtained from the subject. Alternatively, the change in the level of the subfactor may be a decrease in the level of or an absence of the subfactor in the sample obtained from the subject. The change in the level of the subfactor may be a decrease or a down-regulation of the expression or activity of the subfactor in the sample obtained from the subject.

a. Factor

The method can identify one, two, three, or more factors for enhancing lipid metabolism alone or in combination in the sample obtained from the subject in need thereof. The method can measure or detect the change in the level of the factor in the sample alone, in combination with one, two, three, or more factors, in combination with one, two, three, or more subfactors, or any combination thereof.

The factor may be a nucleic acid sequence, an amino acid sequence, or a combination thereof. The nucleic acid sequence may be DNA, RNA, cDNA, a variant thereof, a fragment thereof, or a combination thereof. The amino acid sequence may be a protein, a peptide, a variant thereof, a fragment thereof, or a combination thereof.

(1) Nrf2/SKN-1

The factor may be Nrf2/SKN-1. SKN-1 is the C. elegans ortholog of mammalian Nrf2. Nrf2/SKN-1 is a transcription factor that regulates oxidative stress responses and longevity. Gain-of-function mutations in SKN-1 may induce a starvation-like state. As described herein, Nrf2/SKN-1 may also regulate lipid metabolism. Nrf2/SKN-1 regulation of lipid metabolism may be mediated by or dependent upon MDT-15, which is described below. In particular, Nrf2/SKN-1 may regulate lipid metabolism by upregulating the depletion and/or mobilization of stored fat or lipids.

Nrf2/SKN-1 may promote usage of stored fats in response to food deprivation such as fasting and starvation. Nrf2/SKN-1 may promote usage of stored fats in a diet-dependent manner. Nrf2/SKN-1 may promote usage of stored fats in the presence of a high carbohydrate diet. Nrf2/SKN-1 may promote usage of stored fats when amino acid catabolism is repressed or otherwise inactivated.

Nrf2/SKN-1 activity (e.g., constitutive activity) may prevent or decrease accumulation of fat. In particular, Nrf2/SKN-1 activity may prevent or decrease accumulation of fat in the presence of a high carbohydrate diet.

(2) MDT-15

The factor may be MDT-15. MDT-15 is a transcriptional regulator of lipid metabolism and interacts with Nrf2/SKN-1.

b. Subfactor

The method can identify one, two, three, or more subfactors alone, in combination, or in combination with the factors described above. The method may measure or detect the change in the level of the subfactor alone, in combination with one, two, three, or more subfactors, in combination with one, two, three, or more factors, or any combination thereof.

The subfactor may be a nucleic acid sequence, an amino acids sequence, or a combination thereof. The nucleic acid sequence may be DNA, RNA, cDNA, a variant thereof, a fragment thereof, or a combination thereof. The amino acid sequence may be a protein, a peptide, a variant thereof, a fragment thereof, or a combination thereof.

(1) Fil-1

The subfactor may be fasting-induced lipase-1 (fil-1). Fil-1 levels may be increased in the sample obtained from the subject relative to the control sample, thereby identifying fit-1 as a subfactor for enhancing lipid metabolism in the subject. Fil-1 mRNA levels may be increased about 1.2-fold to about 4.0-fold, or about 1.5-fold to about 3.0-fold, or about 2-fold in the sample obtained from the subject. In other embodiments, fil-1 mRNA levels may be increased about 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, or 4.0-fold in the sample obtained from the subject.

(2) Fatty Acid Oxidation (FAO) Enzyme

The subfactor may a fatty acid oxidation (FAO) enzyme. Under fasting conditions, fat is utilized through the activation of mitochondrial and peroxisomal fatty oxidation (FAO). These FAO pathways are mediated by a number of enzymes, namely FAO enzymes.

The FAO enzyme may be acs-3, acs-11, cpt-1, cpt-3, cpt-4, cpt-5, F08A8.2, acdh-7, ech-8, GCLC, GCLM, NQO1, ACOX1, ACOX2, CPT1, or CPT2.

An mRNA level of one or more of the FAO enzymes acs-1, acs-3, acs-11, cpt-1, cpt-3, cpt-4, cpt-5, cpt-6, acox-1, F08A8.2, F08A8.3, F08A8.4, acdh-1, acdh-7, B0272.3, ech-1, ech-4, B0303.3, F53A2.7, and ech-8 may be increased about 1.2-fold to about 4.0-fold or about 1.5-fold to about 3.0-fold in the sample obtained from the subject. In other embodiments, the mRNA level of one or more of the FAO enzymes acs-1, acs-3, acs-11, cpt-1, cpt-3, cpt-4, cpt-5, cpt-6, acox-1, F08A8.2, F08A8.3, F08A8.4, acdh-1, acdh-7, B0272.3, ech-1, ech-4, B0303.3, F53A2.7, and ech-8 may be increased about 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, or 4.0-fold in the sample obtained from the subject.

In still other embodiments, the increased mRNA level of one or more of the FAO enzymes acs-3, acs-11, cpt-1, cpt-3, cpt-4, acdh-7, and ech-8 may be dependent upon Nrf2/SKN-1, which is described above. The increased mRNA level of cpt-5 and F08A8.2 may not be dependent upon Nrf2/SKN-1.

The mRNA level of one or more FAO enzymes acs-18, C48B4.1, acdh-2, acdh-8, hacd-1, ech-7, and ech-9 may be decreased by about 10% to about 90% or about 25% to about 75% in the sample obtained from the subject. In other embodiments, the mRNA level of one or more FAO enzymes acs-18, C48B4.1, acdh-2, acdh-8, hacd-1, ech-7, and ech-9 may be decreased by about 10%, 15%, 20%, 25%, 30%, 35%, 40%, 45%, 50%, 55%, 60%, 65%, 70%, 75%, 80%, 85%, or 90% in the sample obtained from the subject.

Expression of GCLC, GCLM, NQO1, ACOX1, ACOX2, CPT1, and CPT2 may be dependent upon Nrf2/SKN-1 in mammalian cells, for example, human cells.

### 3. Method of Treatment and/or Protection

Also provided herein is a method for treating a disease in a subject in need thereof, the method comprising administering to the subject an agent capable of increasing Nrf2/SKN-1 activity, MDT-15 activity, or a combination thereof in the subject. Altering (e.g., increasing) the level or activity of one or more of the factors described above may protect the subject from the disease. Altering the level or activity of one or more of the subfactors described above may protect the subject from the disease.

The disease may be obesity, heart disease, or a metabolic disorder associated with deregulated lipid homeostasis. The disease may be obesity.

a. Agent

In the subject suffering from the disease, the agent may alter the level or activity of one or more of the factors discussed above in the subject such that the level or activity leads to increased utilization of lipids, fat, stored lipids, and/or stored fat and/or prevents accumulation of lipids and/or fat in the subject. The agent may alter the level or activity of one or more of the subfactors discussed above in the subject such that the level or activity leads to increased utilization of lipids, fat, stored lipids, and/or stored fat and/or prevents accumulation of lipids and/or fat in the subject. In some embodiments, the agent may enhance utilization of stored lipids or fat in the subject.

### 4. Method of Monitoring Efficacy of Treatment

Also provided herein is a method of monitoring an efficacy of treatment of the disease in a subject undergoing treatment of the disease in any form. In some embodiments, the treatment may be a fasting diet. The method of monitoring may apply the method of identifying factors and subfactors for enhancing lipid metabolism described above to determine if the treatment of the disease has a therapeutic effect in the subject. The disease may be obesity, heart disease, or a metabolic disorder associated with deregulated lipid homeostasis. The disease may be obesity.

The method of monitoring may include obtaining a first sample from the subject before treatment has begun and a second sample from the subject after treatment has begun. The levels of one or more factors can be measured or detected in the first and second samples to determine a first level and a second level of the one or more factors, respectively. The first and second levels of the one or more factors may be compared to determine if the second level is different or changed (e.g., higher or lower) from the first level, in which the difference indicates whether the disease treatment has had a therapeutic effect in the subject.

The method of monitoring may also include measuring or detecting first and second levels of one or more subfactors in the first and second samples, respectively, and comparing the first and second levels of the one or more subfactors. If the second level of the one or more subfactors is different or changed (e.g., higher or lower) from the first level, the difference then further indicates whether the disease treatment has had a therapeutic effect in the subject.

In some embodiments, the method of monitoring may include monitoring the efficacy of a treatment for obesity in the subject. Such a method may include measuring the first level of Nrf2/SKN-1 in the first sample obtained from the subject before the onset of treatment and measuring the second level of Nrf2/SKN-1 in the second sample obtained from the subject after the onset of treatment. This method may also include comparing the measured second level of Nrf2/SKN-1 to the measured first level of Nrf2/SKN-1 and determining if the treatment is effective if the second level of Nrf2/SKN-1 is greater than the first level of Nrf2/SKN-1 or that the treatment is not effect if the second level of Nrf2/SKN-1 is equal or less than the first level of Nrf2/SKN-1.

### 5. Kit

Also provided herein is a kit for use with the methods disclosed herein. The kit can include one or more reagents for detecting the factors and subfactors either alone or in any combination thereof. The one or more reagents may be any of those reagents known in the art for immunoassays (e.g., ELISA, western blotting, immunoprecipitation (IP), immunohistochemistry, etc.) to detect the factors and subfactors. The one or more reagents can also be any of those reagents known in the art for detecting nucleic acids, for example, but not limited to, polymerase chain reaction (PCR), reverse-transcriptase-PCR (RT-PCR), northern blotting, quantitative PCR (qPCR), and so forth. The kit may also include one or more controls and instructions for how to use the kit.

The present invention has multiple aspects, illustrated by the following non-limiting examples.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### Examples

Animals maintain metabolic homeostasis through the coordinated metabolism of available intracellular nutrients. When fasted, animals switch their utilization of dietary carbohydrates to stored fats and proteins to satisfy energy requirements¹. However, it remains elusive whether and how animals coordinate the use of lipids and amino acids during starvation. SKN-1 is the C. elegans ortholog of mammalian Nrf2, an evolutionarily conserved transcription factor that regulates oxidative stress responses² and longevity³⁻⁶. Here, we show that during starvation the metabolism of amino acids and lipids are transcriptionally coordinated by SKN-1/Nrf2. In response to fasting, C. elegans with compromised amino acid catabolism display accelerated mobilization of stored fat and enhanced expression of genes in fatty acid oxidation. Notably, this phenotype is dependent on the type of food ingested prior to fasting indicating that diets can predetermine future metabolic adaption responses during food deprivation. Defective amino acid catabolism when coupled to starvation leads to hyper SKN-1 activation, which mediates this lipid metabolism response. Constitutive SKN-1 activation is capable of inducing a similar transcriptional response, which remarkably protects animals from fat accumulation when fed a high carbohydrate diet. In human cells, Nrf2 activity is similarly coupled to the expression of fatty acid oxidation genes. Our findings identify a novel mechanism for coordinating the metabolism of lipids and amino acids, and implicate SKN-1/Nrf2 as a potential therapeutic target for obesity.

Amino acids are catabolized as fuel during starvation. In particular, proline, arginine and ornithine are catabolized through common metabolic pathways and are preferentially utilized by animals in response to acute food deprivation, as evidenced by their early and significant reduction in under fasting conditions⁷. alh-6 encodes the C. elegans 1-pyrroline-5-carboxylate dehydrogenase (P5CDH)⁸, an evolutionarily conserved mitochondrial enzyme involved in proline, arginine and ornithine catabolism. Thus, alh-6 mutation represents a metabolic state defined by compromised catabolism of amino acids central for the fasting response. We hypothesized that during starvation the use of amino acid and lipid are functionally linked, thus prompting an investigation of the effects of alh-6 mutations on fat metabolism. C. elegans is an established model for studying conserved pathways that govern lipid metabolism⁹⁻¹². When fed the standard E. coli OP50 diet ad libitum, wild type and alh-6 mutant worms store similar levels of intestinal fat as measured by Nile Red staining (FIG. 1a, 1b) or Oil Red O staining. However, within a short 3-hour exposure to starvation, alh-6 mutants rapidly mobilized intestinal lipids as compared to wild type worms, which had not measurably utilized these nutrient stores (FIG. 1a, 1b). Following a long-term starvation period, alh-6 mutants continued the hypermobilization of intestinal fat when compared to wild type animals, which also significantly depleted stored lipids (FIG. 1a, 1b). Thus, alh-6 mutants enhance the mobilization of stored fat in response to food deprivation.

We next examined the expression of genes in lipid metabolism. Under starvation conditions, lipid synthesis is halted via transcriptional repression of fatty acid synthesis enzymes. The expression of fatty acid synthesis enzymes were comparably inhibited in wild type and alh-6 mutant worms in response to fasting, indicating that the enhanced depletion of fat was not regulated at the level of de novo synthesis. The fasting-induced lipase-1 (fil-1) is a key component of the C. elegans starvation response¹². Consistent with the enhanced fat mobilization, alh-6 mutants exhibited a significant increase in the expression of fil-1 (FIG. 1c).

Under fasted conditions, fat is utilized through the activation of mitochondrial and peroxisomal fatty acid oxidation (FAO)¹³⁻¹⁶. We found that alh-6 mutants showed increased expression of FAO enzymes specifically under fasted but not well-fed conditions (FIG. 1d, 1e). These enzymes constitute several main steps of mitochondrial and peroxisomal FAO pathways¹⁷, indicating an overall increase of FAO. Thus, alh-6 mutants when fasted, induce enhanced lipid utilization characterized by upregulation of genes in lipolysis and FAO but not de novo lipogenesis.

We noted that while some FAO enzymes were upregulated by fasting in wild type worms, some FAO genes were surprisingly inhibited during starvation, indicating they may not be generally essential for the physiologic response to fasting, but rather specifically respond to the fasted state in the context of the alh-6 mutant background, when amino acid metabolism is compromised. We propose that C. elegans utilize unique FAO enzymes in response to distinct metabolic stress conditions; some metabolic enzymes can have overlapping functions, can be activated in response to specific cellular needs, and/or become activated in response to the severity of the levels of metabolic homeostatic imbalance.

As mutations in alh-6 cause premature aging in a diet-dependent manner⁸, we asked if the enhanced lipid utilization phenotype identified above was also dependent on diet. Remarkably, the enhanced lipid utilization phenotype was abrogated when alh-6 mutants were raised on another common C. elegans diet, the E. coli K-12 strain HT115. Specifically, on this dietary regimen, alh-6 mutants exhibited comparable levels of fat utilization in response to fasting (FIG. 1f, 1g) and showed no significant changes in the expression of FAO genes, when compared to wild type controls. Thus, the diet ingested prior to starvation establishes an organisms' metabolic adaptation program during food deprivation. Supporting this idea, the diet consumed prior to fasting can have significant effects on mouse behavior during food deprivation¹⁸, which suggests that dietary pre-determination of the adaptive response to starvation is evolutionary conserved.

The increased expression of FAO genes in fasted alh-6 mutants indicates the existence of a transcriptional response that monitors and responds to perturbations in these central facets of cellular energy metabolism. A role for the transcription factor SKN-1 has been documented under conditions of oxidative stress and lifespan extension where nutrient availability is either perceived as reduced or actually is reduced²⁻⁶. Furthermore, we recently found that that gain-of-function mutations in skn-1 induce a starvation-like state¹⁹. As such, we proposed that a SKN-1 mediated transcriptional program could mechanistically link amino and fatty acid metabolism. Under well-fed conditions, the expression of the SKN-1 transcriptional activity reporter gst-4p::GFP was similar between wild type and alh-6 mutant L4 worms (FIG. 2a). However, when starved, the SKN-1 reporter was dramatically activated in alh-6 mutants, but not in wild type controls (FIG. 2a). Furthermore, loss of SKN-1 function, either through RNAi depletion or null mutation, substantially reduced the fasting-dependent activation of the SKN-1 reporter in alh-6 mutants (FIG. 2b, 2c). We conclude that impaired amino acid metabolism in alh-6 mutants activates SKN-1 during food deprivation.

We next asked if SKN-1 mediated the enhanced utilization of stored lipids in fasted alh-6 mutants. We found that, seven out of nine FAO genes with increased expression in the fasted alh-6 mutants were no longer upregulated in the absence of SKN-1 (FIG. 2d). Moreover, six of these seven SKN-1 dependent genes contain three to six conserved SKN-1 binding sites^{2,20} in their promoters, indicating that they may be directly regulated by SKN-1. The expression of the carnitine palmitoyl transferase (CPT) gene cpt-5 and the peroxisomal acyl-coenzyme A oxidase gene F08A8.2 were activated independently of SKN-1 in the alh-6 mutants during fasting (FIG. 2e), indicating the existence of other compensatory pathway(s) that function in parallel to SKN-1. Most importantly, mutation of skn-1 abrogated the enhanced depletion of intestinal lipid stores observed in alh-6 mutant worms after fasting (FIG. 2f, 2g), indicating an essential role for SKN-1 in mediating this fasting response.

Subsequently, we asked if skn-1 gain-of-function mutants induce a similar transcriptional response, and more importantly, if they display a change in stored lipids. Consistent with our previous observation that ad libitum fed skn-1 gain-of-function animals behave as if they are starved¹⁹, skn-1 gain-of-function mutant worms upregulated a large number of FAO genes (FIG. 3a), 83% (15/18) of which contain at least one SKN-1 binding site in their promoters. There was significant overlap between the genes upregulated in the skn-1 gain-of-function mutants and those activated in the alh-6 mutants during fasting. This gene expression pattern indicates a SKN-1 dependent pathway for inducing an organism-level metabolic response that is defined by the activation of fatty acid utilization pathways in skn-1 gain-of-function mutants, similar to the alh-6 mutants under conditions of fasting. We then measured the fat content of those gain-of-function mutant worms. Although transcriptionally poised for increased oxidation of stored fat, well-fed skn-1 gain-of-function mutant animals exhibited relatively similar levels of fat content compared to well-fed wild type controls as measured by Nile red staining (FIG. 3b, 3c), and a minor decrease of fat as revealed by Oil Red O staining We hypothesized that the induction of FAO enzymatic activity in mutants with constitutive SKN-1 activation might only significantly impact lipid homeostasis at the organismal level under conditions of metabolic stress. We thus examined the function of constitutively activated SKN-1 in animals fed a high carbohydrate diet (HCD)²¹, which serves as model that mimics the diet-induced obesity observed in mammals. We found that addition of 2% glucose to the standard diet could significantly induce a 250% increase in stored intestinal fat in wild type C. elegans, as compared to worms feeding on a normal diet (FIG. 3b, 3c). Strikingly, when skn-1 gain-of-function mutants were fed the HCD, they did not manifest this increased lipid phenotype (FIG. 3b, 3c). These data suggest that constitutive SKN-1 activation can transcriptionally predispose animals to successfully cope with dietary insults and that this adaptive capacity is capable of suppressing the lipid accumulation phenotype resulting from a HCD.

To identify possible co-regulators of SKN-1 in modulating lipid metabolism, we screened an RNAi library targeting all annotated transcription factors in C. elegans, looking for suppression of the SKN-1 reporter activation observed in the skn-1 gain-of-function mutants¹⁹. We discovered mdt-15 was required for SKN-1 reporter activation, as RNAi targeting mdt-15 significantly abolished the reporter activation (FIG. 3d). Moreover, in a complementary approach, we performed a classical EMS mutagenesis screen for suppressors of the SKN-1 reporter activation in the skn-1 gain-of-function mutant background. We isolated a single complementation group that mapped to the center of LGIII and identified a Gly to Glu mutation in MDT-15. MDT-15 is a transcriptional regulator of lipid metabolism²² and has been found to physically interact with SKN-1²³. Similarly, the activation of the SKN-1 reporter in fasted alh-6 mutants also required mdt-15 (FIG. 3e), whereas nhr-49, another critical lipid transcriptional regulator^{17,24}, is dispensable for such activation. We then tested the role for MDT-15 in SKN-1 mediated lipid metabolism by using mdt-15 RNAi. Since changes in lipid phenotypes were not observed in alh-6 mutants fed the RNAi strain HT115 E. coli diet (FIG. 1f, 1g), we tested the effect of mdt-15 RNAi on lipid gene expression in the skn-1 gain-of-function mutants. These mutants also display enhanced expression of FAO genes when raised on the RNAi bacteria HT115 (FIG. 3f). However it is notable that the changes observed are not identical to those when animals were fed the OP50 E. coli B diet (FIG. 3a), further suggesting a diet dependent response of SKN-1 function in lipid metabolism. RNAi knockdown of mdt-15 largely abolished the effects of skn-1 gain-of-function mutation on FAO gene expression (FIG. 3f), suggesting MDT-15 is a critical cofactor for the transcription of these targets. Together, our results refine the molecular mechanisms by which SKN-1 and MDT-15 cooperate to maintain lipid homeostasis and define MDT-15 as a co-regulator of SKN-1-dependent expression of FAO pathways.

Nrf2 activity has been linked to cancer cell metabolism and lipid biosynthesis in rodents²⁵; however, a role for human Nrf2 in regulating cellular fatty acid oxidation has not been established. As such, we examined if modulating Nrf2 levels could impact FAO gene expression in human cells. RNAi mediated knockdown of Nrf2 inhibited the expression of canonical Nrf2 target genes in human 293T cells (FIG. 3g). Strikingly, knockdown of Nrf2 also significantly reduced the expression of several FAO genes in human cells (FIG. 3h). These data implicate that the SKN-1/Nrf2 regulatory axis may be an ancient pathway for regulating FAO, which is functionally conserved from invertebrates to humans.

Molecular mechanisms that link glucose and lipid metabolic pathways have been previously documented^{26,27}. In this study, we reveal a functional link between amino and fatty acid metabolism, and identify SKN-1 as a transcriptional switch that coordinates the utilization of these two critical nutrients. During food deprivation, fatty acids and amino acids comprise the major cellular energy resources. However, if amino acid metabolism is compromised, organisms respond to this defect by switching on a SKN-1 and MDT-15 mediated transcription program that further activates FAO. We find that constitutively activated SKN-1 can significantly protect animals against the increased lipid storage phenotype when fed a HCD. This finding implicates SKN-1/Nrf2 as a potential target for the treatment of obesity and related metabolic diseases.

### Methods Summary

C. elegans were cultured using standard techniques at 20° C.²⁸. Animals are fed on E. coli OP50 or RNAi inducing bacterial E. coli HT115. For fat staining, animals with indicated treatments were collected, fixed, and stained by Nile red or Oil Red O. Starvation assays were performed in liquid culture. Human embryonic kidney cells 293T were used for mammalian conservation experiments. Cells were transfected with Nrf2 siRNA and collected for further analysis. Detailed methods are outlined herein.

### REFERENCES

1. Cahill, G. F., Jr. Fuel metabolism in starvation. Annu. Rev. Nutr. 26, 1-22 (2006).
2. An, J. H. & Blackwell, T. K. SKN-1 links C. elegans mesendodermal specification to a conserved oxidative stress response. Genes Dev. 17, 1882-1893 (2003).
3. Tullet, J. M. et al. Direct inhibition of the longevity-promoting factor SKN-1 by insulin-like signaling in C. elegans. Cell 132, 1025-1038 (2008).
4. Wang, J. et al. RNAi screening implicates a SKN-1-dependent transcriptional response in stress resistance and longevity deriving from translation inhibition. PLoS. Genet. 6, (2010).
5. Li, X. et al. Specific SKN-1/Nrf stress responses to perturbations in translation elongation and proteasome activity. PLoS. Genet. 7, e1002119 (2011).
6. Bishop, N. A. & Guarente, L. Two neurons mediate diet-restriction-induced longevity in C. elegans. Nature 447, 545-549 (2007).
7. Felig, P., Owen, O. E., Wahren, J. & Cahill, G. F., Jr. Amino acid metabolism during prolonged starvation. J. Clin. Invest 48, 584-594 (1969).
8. Pang, S. & Curran, S. P. Adaptive Capacity to Bacterial Diet Modulates Aging in C. elegans. Cell Metab 19, 221-231 (2014).
9. Ashrafi, K. et al. Genome-wide RNAi analysis of Caenorhabditis elegans fat regulatory genes. Nature 421, 268-272 (2003).
10. O'Rourke, E. J. & Ruvkun, G. MXL-3 and HLH-30 transcriptionally link lipolysis and autophagy to nutrient availability. Nat. Cell Biol. 15, 668-676 (2013).
11. Watts, J. L. Fat synthesis and adiposity regulation in Caenorhabditis elegans. Trends Endocrinol. Metab 20, 58-65 (2009).
12. Jo, H., Shim, J., Lee, J. H., Lee, J. & Kim, J. B. IRE-1 and HSP-4 contribute to energy homeostasis via fasting-induced lipases in C. elegans. Cell Metab 9, 440-448 (2009).
13. Kersten, S. et al. Peroxisome proliferator-activated receptor alpha mediates the adaptive response to fasting. J. Clin. Invest 103, 1489-1498 (1999).
14. Vega, R. B., Huss, J. M. & Kelly, D. P. The coactivator PGC-1 cooperates with peroxisome proliferator-activated receptor alpha in transcriptional control of nuclear genes encoding mitochondrial fatty acid oxidation enzymes. Mol. Cell Biol. 20, 1868-1876 (2000).
15. Herzig, S. et al. CREB controls hepatic lipid metabolism through nuclear hormone receptor PPAR-gamma. Nature 426, 190-193 (2003).
16. Wolfrum, C., Asilmaz, E., Luca, E., Friedman, J. M. & Stoffel, M. Foxa2 regulates lipid metabolism and ketogenesis in the liver during fasting and in diabetes. Nature 432, 1027-1032 (2004).
17. Van Gilst, M. R., Hadjivassiliou, H. & Yamamoto, K. R. A Caenorhabditis elegans nutrient response system partially dependent on nuclear receptor NHR-49. Proc. Natl. Acad. Sci. U.S.A 102, 13496-13501 (2005).
18. Lavin, D. N. et al. Fasting induces an anti-inflammatory effect on the neuroimmune system which a high-fat diet prevents. Obesity. (Silver. Spring) 19, 1586-1594 (2011).
19. Paek, J. et al. Mitochondrial SKN-1/Nrf mediates a conserved starvation response. Cell Metab 16, 526-537 (2012).
20. Blackwell, T. K., Bowerman, B., Priess, J. R. & Weintraub, H. Formation of a monomeric DNA binding domain by Skn-1 bZIP and homeodomain elements. Science 266, 621-628 (1994).
21. Schulz, T. J. et al. Glucose restriction extends Caenorhabditis elegans life span by inducing mitochondrial respiration and increasing oxidative stress. Cell Metab 6, 280-293 (2007).
22. Taubert, S., Van Gilst, M. R., Hansen, M. & Yamamoto, K. R. A Mediator subunit, MDT-15, integrates regulation of fatty acid metabolism by NHR-49-dependent and -independent pathways in C. elegans. Genes Dev. 20, 1137-1149 (2006).
23. Goh, G. Y. et al. The conserved Mediator subunit MDT-15 is required for oxidative stress responses in Caenorhabditis elegans. Aging Cell 13, 70-79 (2014).
24. Van Gilst, M. R., Hadjivassiliou, H., Jolly, A. & Yamamoto, K. R. Nuclear hormone receptor NHR-49 controls fat consumption and fatty acid composition in C. elegans. PLoS. Biol. 3, e53 (2005).
25. Sykiotis, G. P., Habeos, I. G., Samuelson, A. V. & Bohmann, D. The role of the antioxidant and longevity-promoting Nrf2 pathway in metabolic regulation. Curr. Opin. Clin. Nutr. Metab Care 14, 41-48 (2011).
26. Mitro, N. et al. The nuclear receptor LXR is a glucose sensor. Nature 445, 219-223 (2007).
27. Herman, M. A. et al. A novel ChREBP isoform in adipose tissue regulates systemic glucose metabolism. Nature 484, 333-338 (2012).
28. Brenner, S. The genetics of Caenorhabditis elegans. Genetics 77, 71-94 (1974).

### FIGURE LEGENDS

FIG. 1a-1g Mutation in alh-6 induces enhanced fat mobilization and expression of FAO genes during starvation. FIGS. 1a, 1b, Nile red staining of OP50 fed wild type and alh-6 mutants in response to fasting. The representative images are shown in (FIG. 1a) and quantitative data are shown in (FIG. 1b) (n=12 for 0 hours of wild type and alh-6 (lax105) mutants, n=13 for 3 hours of alh-6 mutants, n=9 for other groups). FIG. 1c, Expression of fil-1 (n=3). FIGS. 1d, 1e, Expression of FAO genes under fasted (FIG. 1d) and well-fed (FIG. 1e) conditions (n=3). FIGS. 1f, 1g, Nile red staining of HT115 feeding wild type and alh-6 mutants in response to fasting. The representative images are shown in (FIG. 1f) and quantitative data are shown in (FIG. 1g) (n=8 for fed wild type, n=9 for fed alh-6 mutants and fasted wild type, n=10 for fasted alh-6 mutants). *p<0.05, **p<0.01, ***p<0.001, versus wild type controls under same treatment unless specifically indicated.

FIGS. 2a-2g SKN-1 mediates the effects of alh-6 mutation on lipid metabolism during starvation. FIG. 2a, Mutation of alh-6 activates gst-4p::GFP, a SKN-1 transcriptional activity reporter, during fasting. FIGS. 2b, 2c, RNAi mediated knockdown (FIG. 2b) or mutation (FIG. 2c) of skn-1 abolished the activation of gst-4p::GFP. Presence of the skn-1 balancer is indicated by GFP expression in pharynx as pointed out by arrow. FIGS. 2d, 2e, The increased expression of FAO genes in fasted alh-6 mutant is either dependent (FIG. 2d) or independent (FIG. 2e) on skn-1 (n=3). FIGS. 2f, 2g, Nile red staining of worms with indicated genotypes under fasted condition. The representative images are shown in (FIG. 2f) and quantitative data are shown in (FIG. 2g) (n=11 for wild type, n=10 for other groups). *p<0.05, **p<0.01, ***p<0.001, versus wild type controls under same treatment unless specifically indicated.

FIGS. 3a-3h Constitutive activation of SKN-1 protects animals from HCD induced fat accumulation. FIG. 3a, Expression of FAO genes those are regulated by skn-1 (lax188) gain-of-function mutation (n=3). FIGS. 3b, 3c, Nile red staining of worms with indicated genotypes fed OP50 or OP50 plus 2% glucose. The representative images are shown in (FIG. 3b) and quantitative data are shown in (FIG. 3c) (n=5 for wild type fed OP50, n=10 for wild type fed OP50 plus 2% glucose, n=8 for skn-1 (lax188) fed OP50, n=7 for other groups). FIGS. 3d, 3e, Knockdown of mdt-15 abolishes the activation of gst-4p::GFP in skn-1 gain of function mutants (FIG. 3d) or fasted alh-6 mutants (FIG. 3e). FIG. 3f, Expression of FAO genes that are regulated by skn-1 (lax188) fed HT115 bacterial containing L4440 control or mdt-15 RNAi plasmids. (n=2 for skn-1 (lax188) fed control RNAi, n=3 for other groups). FIGS. 3g, 3h, Knockdown of Nrf2 inhibits expression of its canonical target genes (FIG. 3g) and FAO genes expression (FIG. 3h) (n=3 for control, n=5 for Nrf2 siRNA). *p<0.05, **p<0.01, ***p<0.001, versus wild type controls under same treatment unless specifically indicated.

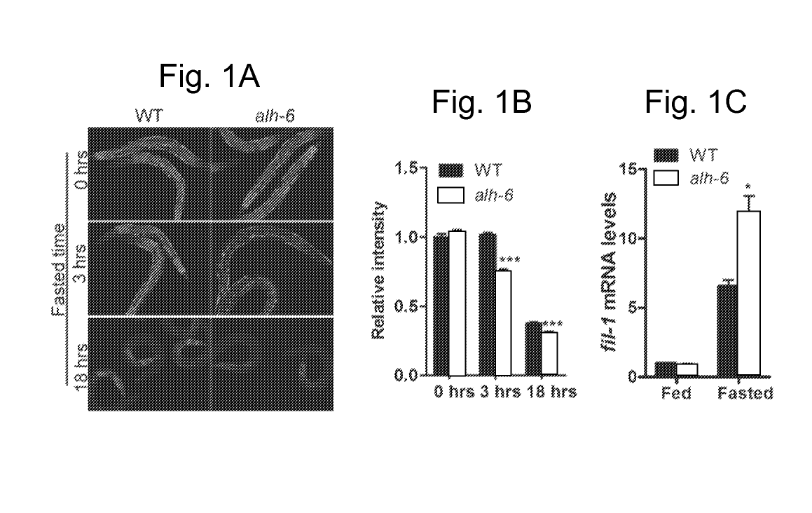

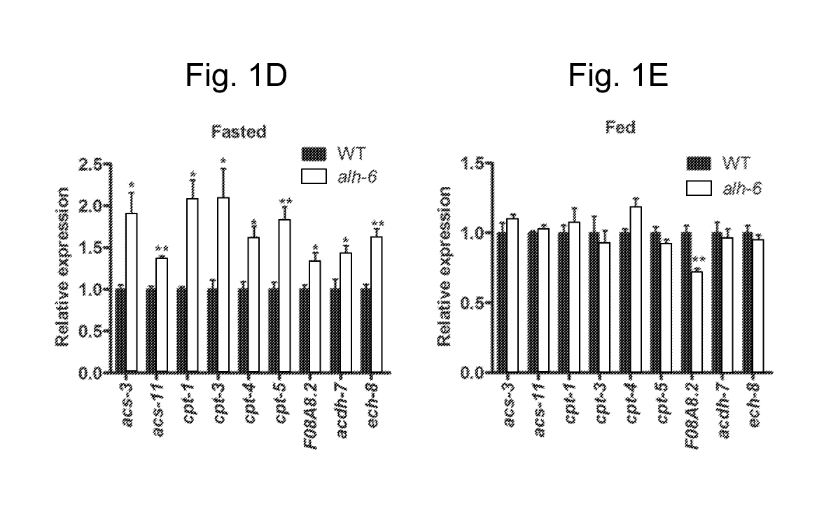

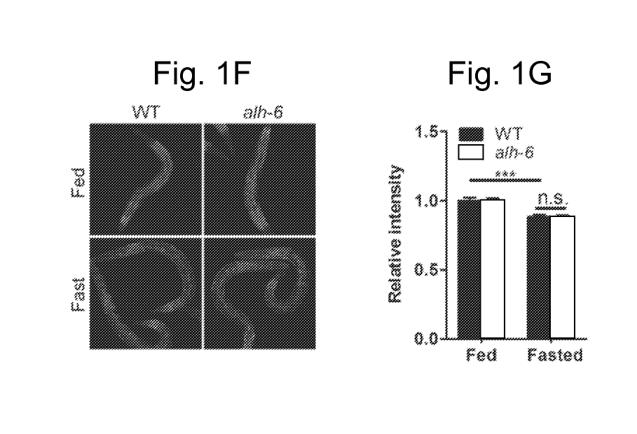

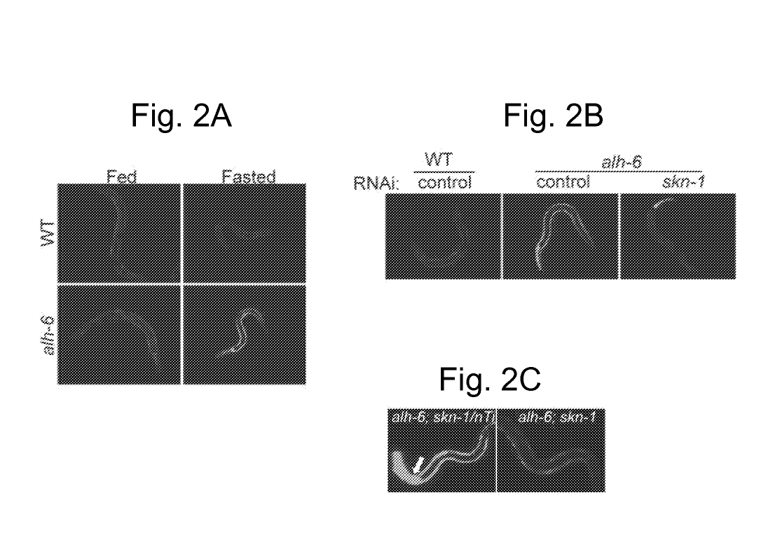

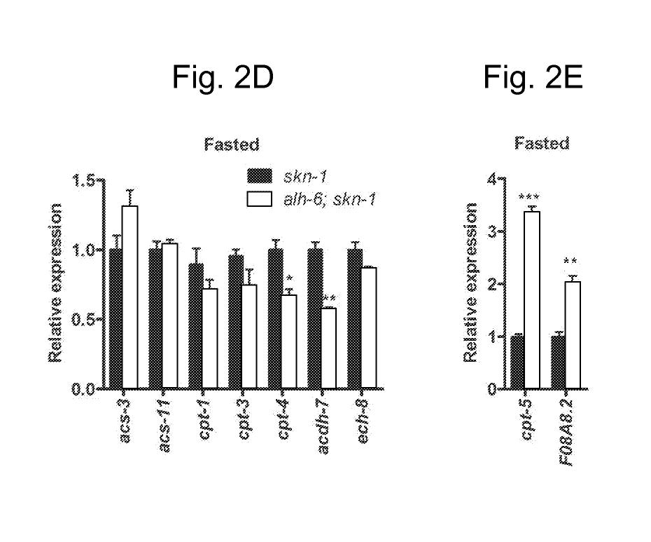

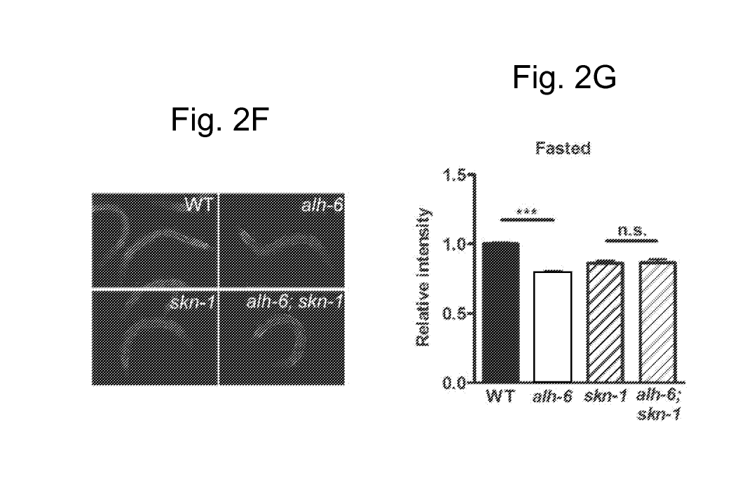

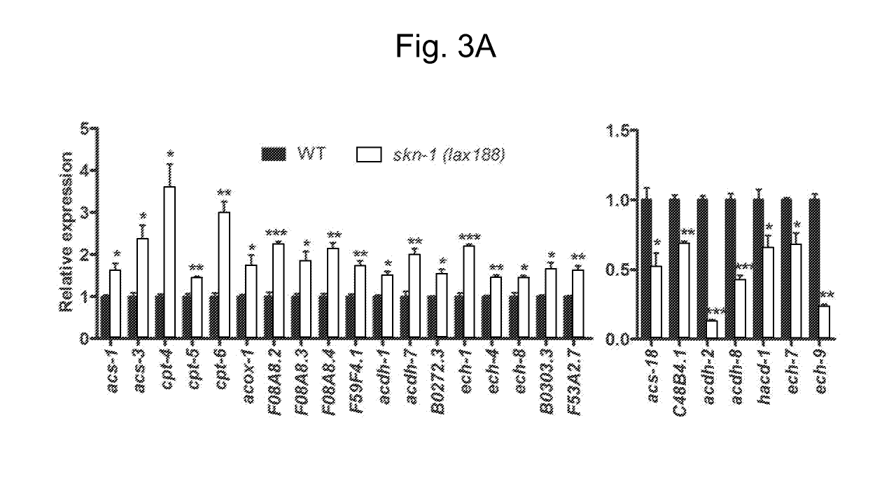

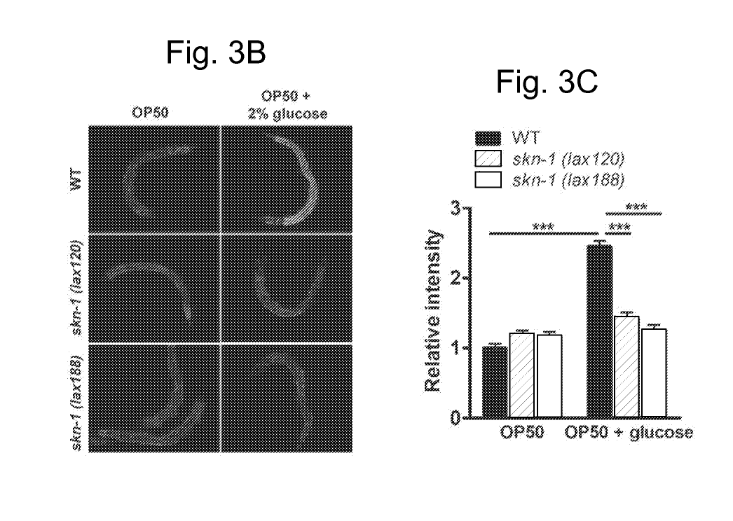

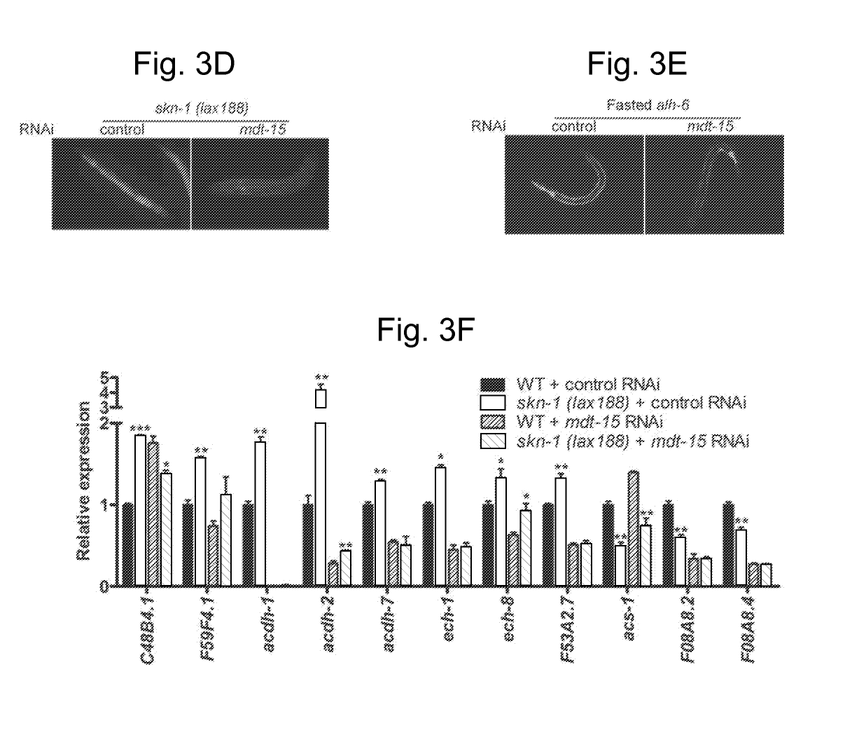

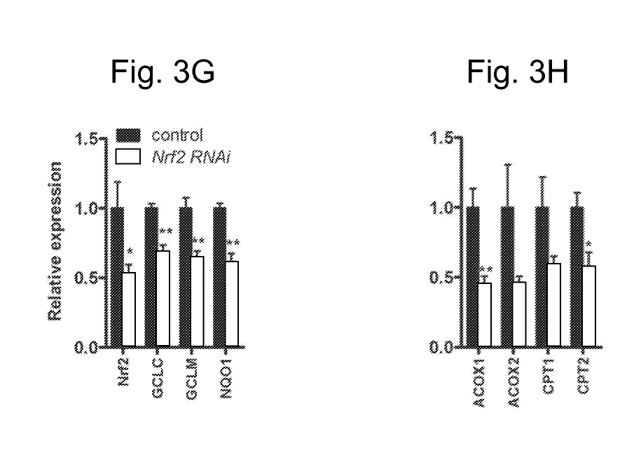

### METHODS

### C. elegans Strains Utilized in this Study

The following strains were used: wild type N2 Bristol, SPC207[skn-1 (lax120)], SPC227[skn-1 (lax188)], SPC321[alh-6 (lax105)], CL2166[gst4-p::gfp], SPC276[skn-1 (lax188), mdt-15 (lax225), gst4-p::gfp], VC1772[skn-1 (ok2315) IV/nTi[gIs51] (IV; V)]. Double mutants were generated by standard genetic techniques.

### Human Cell Culture

293T cells were cultured in DMEM supplemented with 10% fetal bovine serum. At 50-70% confluence, cells were transfected with control or Nrf2 siRNA by using Lipofectamine RNAiMax (Life Technologies). After twenty-four hours, cells were washed and collected in Trizol reagent (Invitrogen) for RNA extraction.

### Starvation Assay

Synchronized L1 animals were added to NGM plates seeded with indicated bacteria. After two days at 20° C., L4 animals were collected, washed with M9 buffer at least three times and then subjected to fasting in M9 liquid with shaking for indicated time before collection for further analysis.

### Nile Red Staining

Nile red staining was performed as previously described²⁹. Briefly, animals of indicated genotypes were collected, fixed in 40% isopropanol, and stained in Nile red working solution in dark for two hours. Then worms were washed with M9 for at least half an hour, mounted on slides and imaged with the green fluorescent protein (GFP) channel of microscope Zeiss Axio Imager with Zen software package. Fluorescent density was measured by using NIH ImageJ software.

### Oil Red O Staining

Animals of indicated genotypes were collected and fixed in 1% formaldehyde in PBS. Then samples were frozen and thawed three times on a dry ice/ethanol bath. Worms were washed with PBS three times before staining with freshly prepared Oil Red O working solution. After staining for half an hour, worms were washed again for 15 minutes and mounted on slides, and imaged under a bright field illumination.

### RNA Interference Treatment

HT115 bacteria containing specific dsRNA-expression plasmids were seeded on NGM plates containing 5 mM IPTG and RNAi was induced at room temperature for 24 hours. Synchronized L1 animals were added to those plates to knockdown indicated genes.

qRT-PCR

qRT-PCR were performed as previously described⁸. Briefly, worms of the indicated genotype and stages were collected, washed in M9 buffer and then homogenized in Trizol reagent (Life Technologies). RNA was extracted according to manufacturer's protocol. DNA contamination was digested with DNase I (New England Biolabs) and subsequently RNA was reverse-transcribed to cDNA by using the SuperScript® III First-Strand Synthesis System (Life Technologies). Quantitative PCR was performed by using SYBR Green (BioRad). The expression of snb-1 was used to normalize samples.

### Statistical Analysis

Data presented reflect biological replicates as indicated in each sample's n. Sample sizes were determined to reliably reveal the statistic significance given the magnitude of the changes expected in each experiment. No randomization or binning was used. Data were presumed to be normally distributed. Data were presented as mean±SEM. Data were analyzed by using unpaired student t test. P<0.05 was considered as significant.
29. Pino, E. C., Webster, C. M., Carr, C. E. & Soukas, A. A. Biochemical and high throughput microscopic assessment of fat mass in Caenorhabditis elegans. J. Vis. Exp. (2013)

Oil Red O staining of fat content in alh-6 mutants during fasting. a, Schematic of amino acid catabolism pathways regulated by ALH-6. b, Oil Red O staining of OP50 fed wild type and alh-6 mutants in response to three hours of fasting.

Expression analysis of lipid metabolism genes. a, Expression of fatty acid synthesis genes under well-fed or fasted conditions (n=3). b, c, alh-6 dependent FAO genes are either upregulated (b) or downregulated (c) by starvation in wild type worms (n=3). d, Expression of FAO genes under fasted conditions in worms fed HT115 bacteria (n=3). *p<0.05, **p<0.01, ***p<0.001, versus wild type controls under same treatment unless specifically indicated.

Comparison of FAO genes that are deregulated in starved animals with compromised amino acid catabolism or well-fed animals with constitutively activated SKN-1. Genes that are upregulated by impaired amino acid catabolism during fasting are listed in the blue box; genes upregulated in well-fed constitutively activated SKN-1 mutants are listed in the orange box.

Oil Red O staining of fat content in skn-1 gain-of-function mutants. Oil Red O staining of worms with indicated genotypes fed OP50 or OP50 supplemented with 2% glucose.

Effects of mdt-15 and nhr-49 on SKN-1 transcriptional activation. a, mdt-15 mutation abolishes the gst-4p::GFP activation in skn-1 gain-of-function mutants. b, RNAi knockdown of nhr-49 has no effect on gst-4p::GFP activation in alh-6 mutants in response to fasting.

During food deprivation, animals use fat and amino acids as their fuel sources. When amino acid catabolism is compromised due to alh-6 mutation, an adaptive response is triggered, which activates a SKN-1 mediated transcriptional program for the induction of FAO genes to maintain energy homeostasis. Constitutively activated SKN-1 induces similar transcriptional changes in FAO genes that protect animals from diet-induced obesity.

Predicted SKN-1 binding sites in the promoters of FAO genes that are altered in alh-6 mutant animals during fasting.

Conserved SKN-1 binding site WWTDTATC was detected in the promoter region up to 2 kb by using Regulatory Sequence Analysis Tools (RSAT). D: Sense strand, R: antisense strand.

Predicted SKN-1 binding sites in the promoters of FAO genes that are altered in well-fed skn-1 gain-of-function mutants.

Conserved SKN-1 binding site WWTDTATC was detected in the promoter region up to 2 kb by using Regulatory Sequence Analysis Tools (RSAT). D: Sense strand, R: antisense strand.

1. A method for treating obesity in a subject in need thereof, the method comprising administering to the subject an agent capable of increasing Nrf2/SKN-1 activity, MDT-15 activity, or a combination thereof in the subject.

2. The method of claim 1, wherein the agent enhances utilization of stored lipids in the subject.

3. A method for protecting a subject in need thereof from obesity, the method comprising increasing Nrf2/SKN-1 activity, MDT-15 activity, or a combination thereof in the subject.

4. The method of claim 1, further comprising altering the expression of a gene selected from the group consisting of: fil-1, a fatty acid oxidation enzyme, and combinations thereof.

5. The method of claim 4, wherein the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.

6. A method for monitoring an efficacy of a treatment for obesity in a subject, the method comprising:
(a) measuring a first level of Nrf2/SKN-1 in a first sample obtained from the subject before the onset of the treatment;
(b) measuring a second level of Nrf2/SKN-1 in a second sample obtained from the subject after the onset of treatment;
(c) comparing the measured second level of Nrf2/SKN-1 to the measured first level of Nrf2/SKN-1; and
(d) determining that the treatment is effective if the second level of Nrf2/SKIN-1 is greater than the first level of Nrf2/SKN-1 or that the treatment is not effective if the second level of Nrf2/SKN-1 is equal or less than the first level of Nrf2/SKN-1.

7. The method of claim 6, wherein the treatment is a fasting diet.

8. The method of claim 3, further comprising altering the expression of a gene selected from the group consisting of fil-1, a fatty acid oxidation enzyme, and combinations thereof.

9. The method of claim 8, wherein the fatty acid oxidation enzyme is selected from the group consisting of acs-3, acs-11, cpt-1, cpt-3, cpt-4, cpt-5, F08A8.2, acdh-7, ech-8, GCLC, GCLM, NQO1, ACOX1, ACOX2, CPT1, CPT2, and combinations thereof.
